(19) United States
(12) Patent Application Publication
(10) Pub. No.: US 2022/0174892 A1
(43) Pub. Date: Jun. 9, 2022

(54) TREE EGGPLANT AND CULTIVATION METHOD, RAPID PROPAGATION METHOD AND APPLICATION THEREOF

(71) Applicant: Huazhong Agricultural University, Wuhan (CN)

(72) Inventors: Zhibiao YE, Wuhan (CN); Yuyang ZHANG, Wuhan (CN); Junhong ZHANG, Wuhan (CN); Hanxia LI, Wuhan (CN); Chuanding QIN, Wuhan (CN)

(73) Assignee: Huazhong Agricultural University, Wuhan (CN)

(21) Appl. No.: 17/537,533

(22) Filed: Nov. 30, 2021

(30) Foreign Application Priority Data
Dec. 9, 2020 (CN) 202011454792 .1

(51) Int. Cl. A01G 17/00; A01H 1/02; A01G 23/04; A01G 13/02; A01G 24/20

(52) U.S. Cl. CPC A01G 17/00; A01H 1/02; A01G 24/20; A01G 13/0275; A01G 23/04

(57) ABSTRACT

A tree eggplant and a cultivation method, a rapid propagation method and an application thereof are provided by the present disclosure. In the present disclosure, the Solanum wrightii is specifically selected as the female parent, and the Solanum torvum is used as the male parent, and the tree eggplant variety obtained by hybridization has fast growth, developed trunk, strong growth potential, and is perennial, and is strong in photosynthesis, strong in nutrient and water absorption capacity, high in affinity with the scion, fast in healing at the scion interface of the rootstock and good in healing; and has good resistance to soil-borne diseases such as fusarium wilt, verticillium wilt, bacterial wilt and root knot nematode; and the rapid propagation method of the tree eggplant has strong operability and high efficiency, and is easy to popularize in practical application.

### CROSS-REFERENCE TO RELATED APPLICATIONS

The application claims priority to Chinese patent application No. 202011454792. 1, filed on Dec. 9, 2020, the entire contents of which are incorporated herein by reference.

### TECHNICAL FIELD

The present disclosure relates to the technical field of genetics and breeding, and more specifically relates to a tree eggplant and a cultivation method, a rapid propagation method and an application thereof.

### BACKGROUND

Solanaceae plants include eggplant, tomato, pepper, potato and tobacco, are common crops in human life. Wild eggplant is often used as a rootstock in the commercial breeding of Solanaceous plants, including tomato and eggplant, to resist the occurrence of soil-borne pests and diseases.

According to prior art reports, soil infectious bacteria of Solanaceae plants can survive in soil for as long as 5 to 7 years, and are difficult to control by applying pesticides. Therefore, it is of great practical significance to breed Solanaceae rootstocks which can resist insect pests, grow quickly and have high grafting affinity. Grafting rootstock with scion is an economic and effective way to solve the resistance of plant diseases and insect pests in planting process. It not only improves yield, but also improves quality, so it is widely used in vegetable planting.

At present, the breeding of grafted rootstocks has become the breeding goal of eggplant disease resistance breeding at home and abroad. In addition, the amount of grafting work at seedling stage is large, and each crop needs to be grafted once.

Up to now, there have been no reports about the interspecific hybridization of Solanaceae plants and the breeding of high quality Solanaceae rootstock varieties.

### SUMMARY

The present disclosure aims to provide a tree eggplant with fast growth, strong growth, good resistance, high affinity for grafting, and can be perennial. At the same time, the present disclosure provides a cultivation method, a rapid propagation method, and an application of the tree eggplant.

In order to achieve the above object, the present disclosure adopts the following technical solution:

a cultivation method of a tree eggplant, specifically comprising:

taking a Solanum wrightii as a female parent, taking wild a Solanum torvum as a male parent, after hybrid pollination, and in turn after embryo rescue and seedling transplantation, obtaining the tree eggplant.

Specifically, in the prior art, the traditional eggplant rootstock Toru Bam used for seedling grafting had unstable resistance, slow germination, low germination rate, irregular growth, slow seedling growth, long seedling age, and poor grafting affinity. Therefore, it has limitations in large-scale production and application as a grafting rootstock. Therefore, it is urgent to cultivate a rootstock with fast-growing, strong growth, good resistance, and high affinity rootstock to meet the actual production requirements. Through a large number of experimental studies, it is found that Solanum wrightii has a high growth potential. Through interspecific hybridization with Solanum torvum, rootstock varieties with fast growth, strong growth potential, good resistance and perennial can be obtained.

Further, in the above technical solution, the hybrid pollination comprises: making emasculation and then bagging of a flower of the Solanum wrightii female parent 1 to 2 days before opening, selecting pollen of a male flower of the Solanum torvum male parent from 9:00 to 12:00 a.m. at a full opening stage of the male flower of the Solanum torvum male plant.

Further, in the above technical solution, the embryo rescue comprises: taking an immature hybrid young fruit 20 to 25 days after the hybrid pollination, sterilizing with NaClO (Specifically, disinfecting with 70% alcohol for 2 minutes, then disinfecting with 0.6% NaClO for 5 minutes, and cleaning with sterilized distilled water for 3 times), cutting and isolating to obtain young embryo, and transferring to an induced proliferation medium for culture, then transferring induced embryoids to a regeneration medium to obtain regenerated buds, and finally transferring the obtained regenerated buds to a rooting medium to obtain rooting.

Specifically, in the above technical solution, young embryo and hybrid seedlings of the interspecific hybridization are obtained by the method of embryo rescue, and then the stock with fast growth, strong growth and good resistance is cultivated, which is a technical innovation of eggplant stock breeding.

Specifically, the seed abortion is obtained by traditional interspecific hybridization, and the seed quantity is very small, the germination rate is as low as 0.4%, so it is difficult to obtain the interspecific hybridization seed, while the survival rate of embryo rescue is more than 60% by using the above technical scheme to obtain the interspecific hybridization seed.

In detail, in the above technical solution, the induced proliferation medium is MS+2 mg/l ZR, and a subculture temperature and a time are 22 to 28° C. and 30 to 40 days, respectively;

In detail, in the above technical solution, the regeneration medium is MS+2 mg/l 6-BA+0.5 mg/l IAA;

in detail, in the above technical solution, the rooting medium is MS+0.4 mg/l IBA, the rooting time is 20 to 36 days, the rooting rate is 100%.

In detail, in the above technical solution, the seedling transplanting comprises washing a culture medium adhered to a test-tube seedling obtained by culturing and rooting with water, transplanting to seedling-refining substrate, periodically watering nutrient solution, covering with film and moisturizing, during the first week after transplanting, the film is kept at 100% humidity and maintained strong light. After 7 days, the film is gradually removed and then transplanted to the greenhouse.

In detail, in the above technical solution, the seedling-refining substrate is a mixture of coconut bran, vermiculite and perlite in a volume ratio of (0.8-1.2):(0.8-1.2):(0.8-1.2).

In a preferred embodiment of the present disclosure, the cultivation method for fast growth, strong growth and good resistance tree eggplant comprises the following steps:

taking wild Solanum wrightii as female parent and wild Solanum torvum as male parent for interspecific hybridization, female flowers are emasculated and bagged one day before opening to avoid pollination of other pollens. Flowers with open parents are selected from 9:00 to 12:00 a.m. on the pollination day and pollen from anthers is pollinated with tweezers.

(2) 20 to 25 days after hybrid pollination, taking the immature hybrid young fruits and sterilizing with NaClO on surface, cutting the immature hybrid young fruits and isolating to obtain the young embryos with the sterile blade, and transferring to the induced proliferation medium (MS+2 mg/l ZR), and grow at 25±1° C. The induced embryoids are transferred to the regeneration medium (MS+2 mg/l 6-BA+0.5 mg/l IAA) to obtain regenerated buds, and the regenerated buds are transferred to the rooting medium (MS+0.4 mg/l IBA). After 15 to 20 days, the roots are grown and developed roots are formed in 1 to 2 weeks.

(3) Taking out the test tube seedlings cultured in vitro, gently removing the adhering culture medium with distilled water, and transplanting them into the refined seedling substrate composed of coconut bran, vermiculite and perlite with a volume ratio of 1:1:1, irrigating with ½ MS nutrient solution every two weeks, covering the test tube seedlings with fresh film to keep them moist, and maintaining strong light. After 5 to 8 days, the plastic film can be removed gradually, and the small plantlets can be quickly grown into large seedlings in the substrate, and then transplanted to the soil or large seedling bags for growth.

Another aspect of the disclosure provides a tree eggplant cultivated by the cultivation method, which has the features of fast growth, strong growth, good resistance and perennial.

Further, since the tree eggplant can not bear fruit normally and needs asexual reproduction, the present disclosure also provides a rapid propagation method of the tree eggplant with fast growth, strong growth, good resistance and perennial, comprising the following steps:

S1: cutting stem segments with buds of aseptic seedlings of the tree eggplant, inoculating to a multiplication and propagation medium to induce proliferation, and obtaining multiplication and propagation buds;

S2: inoculating the multiplication and propagation buds into a tissue culture medium, then under conditions of 23 to 28° C., 22 to 27 μmol·m⁻²·s⁻¹ and 10 to 13.5 hours of sunlight, obtaining regenerated sterile buds after culturing for 10 to 14 days; and

S3: cutting the regenerated sterile buds and transferring to a sealed tissue culture flask containing a sterile bud rooting medium, placing under natural scattering light, then gradually opening to refine seedlings, and finally transferring to growth substrate for natural growth.

Further, in the above technical solution, the multiplication and propagation medium is MS+2 mg/l ZR, and a length of a shoot segment with the buds is 0.4 to 0.6 cm;

Further, in the above technical solution, the tissue culture medium is MS+7 g agar+25 g sucrose, pH is 5.8 to 6.0.

Further, in the above technical solution, the sterile bud rooting medium is ½ MS+IBA 0.2 mg·L⁻¹, a seedling opening time is at least 3 days, and the growth substrate is a mixture of the perlite with a volume ratio of 1:1 and sterilized and decomposed organic fertilizer.

In another aspect, the disclosure provides an application of the tree eggplant with fast growth, strong growth potential, good resistance and perennial.

Specifically, the application comprises being used as rootstocks for grafting solanaceous plant and perennial cultivation and as landscape trees for perennial growth in afforestation of tropical areas.

In detail, when the above fast-growing, disease-resistant, pest-resistant and perennial tree eggplant is used as rootstocks for grafting Solanaceae plants, the grafting methods mainly include splitting method and bonding method, etc., clean and sharp blades and grafting clips are commonly used in the grafting process; as long as the temperature is appropriate, grafting can be carried out, and the grafting can be carried out in the tropical areas on an annual basis.

After the trunk of the tree eggplant is topped, the side branches produced are topped again, and the sun buds are formed on the side branches as the rootstock interface of grafting; the grafting method is adopted, each branch has multiple ear buds, and after the top of the branch is cut, an ear bud is grafted by the splitting method. When attention needs to be paid, the graft should be sealed with plastic wrap.

In addition, as a high-level grafting of rootstock, the management requirements of the grafted seedlings are not strict with the common seedling grafting method, but in order to improve the grafting survival rate, the grafted seedlings must be well managed.

Compared with the prior art, the benefits of the present disclosure are:

(1) according to the disclosure, Solanum wrightii is specifically selected as the female parent, and Solanum torvum is used as the male parent, so as to hybridize to obtain a variety of tree eggplant, which is fast in growth, strong in growth, good in resistance and perennial, and has good resistance, and can resist soil-borne diseases such as fusarium wilt, verticillium wilt, bacterial wilt and root knot nematode;

(2) the tree eggplant variety obtained by crossing has developed trunk, strong growth potential, strong photosynthesis, strong nutrient and water absorption ability, high affinity, fast healing and good healing of rootstock scion interface;

(3) the rapid propagation method of the tree eggplant variety obtained by the hybrid has strong operability and high efficiency, and is easy to be popularized in practical application; and

(4) the tree eggplant variety obtained by hybridization can be perennial and widely used. The tree eggplant variety can be used as grafting eggplant, tomato, potato, tobacco and other solanaceous plants, and can be used as a landscape tree for greening planting in the tropical areas.

### BRIEF DESCRIPTION OF DRAWINGS

FIG. 1 is a photograph of a regenerated plant of a tree eggplant obtained after hybrid pollination and embryo rescue provided in an embodiment of the present disclosure;

FIG. 2 is a photograph of a tree eggplant obtained by rapid propagation of stem segments provided in an embodiment of the present disclosure;

FIG. 3 is a field and cultivation of a tree eggplant seedling obtained by rapid propagation provided in an embodiment of the present disclosure;

FIG. 4 is site photograph of tree eggplant seedlings obtained by rapid propagation in the greenhouse provided in the embodiment of the present disclosure;

FIG. 5 is a photograph of a robust tree body grown by pruning the tree eggplant obtained by rapid propagation provided in the embodiment of the present disclosure;

FIG. 6 is a site photograph of the tree eggplant provided in the embodiment of the present disclosure being planted in the green belt as a landscape tree species;

FIG. 7 is a site photograph of eggplant high-position grafting with tree eggplant as rootstock provided in embodiments of the present disclosure;

FIG. 8 is a site photograph of the combination of eggplant and tomato grafted with tree eggplant as rootstocks provided in the embodiments of the present disclosure;

FIG. 9 is a field photograph of a tomato seedling in full flowering stage after grafting tomato as a stock in the embodiments of the present disclosure.

### DETAILED DESCRIPTION OF THE EMBODIMENTS

The present disclosure will be further described in detail below in connection with specific embodiments, so that those skilled in the art more clearly understand the present disclosure.

The following embodiments are only intended to describe the contents of the present disclosure, but are not intended to limit the scope of the present disclosure.

If not specifically indicated, the technical means used in the embodiments of the present disclosure are conventional means well known to those skilled in the art.

Based on the embodiments of the present disclosure, all other embodiments obtained by a person of ordinary skill in the art without fall within the scope of protection of the present disclosure without present disclosure.

Embodiments of the present disclosure provide cultivation method of a tree eggplant, specifically comprising the following steps:

(1) The hybrid pollination: taking wild Solanum wrightii as female parent and wild Solanum torvum as male parent for interspecific hybridization, female flowers are emasculated and bagged one day before opening to avoid pollination of other pollens. Flowers with open parents are selected from 9:00 to 12:00 a.m. on the pollination day and pollen from anthers is pollinated with tweezers.

In the embodiment of the disclosure, the female parent of wild Solanum wrightii and the male parent of wild water eggplant are from the key laboratory of the ministry of horticultural plant biology of Huazhong Agricultural University.

(2) The embryo rescue: taking an immature hybrid young fruit 20 to 25 days after the hybrid pollination, disinfecting with 70% alcohol for 2 minutes, then disinfecting with 0.6% NaClO for 5 minutes, and cleaning with sterilized distilled water for 3 times, cutting the immature hybrid young fruits and isolating to obtain the young embryos with the sterile blade, and transferring to the induced proliferation medium (MS+2 mg/l ZR), and grow at 25±1° C. for 30 to 40 days. The induced embryoids are transferred to the regeneration medium (MS+2 mg/l 6-BA+0.5 mg/l IAA) to obtain regenerated buds, and the regenerated buds are transferred to the rooting medium (MS+0.4 mg/l IBA). After 15 to 20 days, the roots are grown and developed roots are formed in 4 weeks.

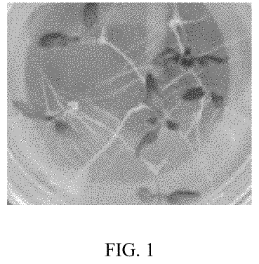

As shown in FIG. 1, the photographs of regenerated plantlets obtained after hybrid pollination and embryo rescue are provided in the embodiments of the present disclosure.

If different hybrid combinations are used, the results are also different, as shown in the table below.

| | Number | | Number of | Survival |
|---|---|---|---|---|
| | of | Fruit | young | rate of |
| | hybrid | setting | embryos | embryo |
| Hybrid combination | flowers | rate (%) | per fruit | rescue (%) |
| Solanum wrightii × | 132 | 3.6 | 5 | 65 |
| Solanum torvum | | | | |
| Solanum torvum × | 125 | 0 | — | — |
| Solanum wrightii | | | | |

If different rooting conditions are adopted, the results will be different, as shown in the table below.

| | | Average | Number | Transplanting |
|---|---|---|---|---|
| Rooting Medium | Rooting | number of | of lateral | survival rate |
| Formula | rate (%) | primary roots | roots | (%) |
| MS | 80 | 4.5 | Less | 90 |
| MS + 0.2 mg/l IBA | 90 | 5.1 | More | 90 |
| MS + 0.4 mg/l IBA | 100 | 5.5 | More | 100 |
| MS + 0.6 mg/l IBA | 90 | 4.6 | Less | 90 |

Seedling transplanting: taking out the test tube seedlings cultured in vitro, gently removing the adhering culture medium with distilled water, and transplanting them into the refined seedling substrate composed of coconut bran, vermiculite and perlite with a volume ratio of 1:1:1, irrigating with ½ MS nutrient solution every two weeks, covering the test tube seedlings with fresh film to keep them moist, and maintaining strong light. After 5 to 8 days, the plastic film can be removed gradually, and the small plantlets can be quickly grown into large seedlings in the substrate, and then transplanted to the soil or large seedling bags for growth.

Compared with the existing cultivar Toru Bam, the tree eggplant cultivated by the above method has the advantages of fast growth, strong growth, good resistance and perennial cultivation, as shown in the table below.

| | | | | | | | Amount of |
|---|---|---|---|---|---|---|---|
| | | | Plant | Stem | | | grafting work |
| | Growth | | height | diameter | Grafted | Affinity | per mu/ |
| | cycle | Resistance | m | cm | plant | % | Labor |
| Tree | Fast and | Soil-borne | 2.5 | 20 | Tomato, | 100 | 0.2 |
| eggplant | orderly | diseases, low | | | Pepper, | | Labor/mu/year |
| | seedling | temperature, | | | Eggplant, | | |
| | emergence | high | | | Potato, | | |
| | | temperature, | | | Tobacco | | |
| | | drought, | | | | | |
| | | saline-alkali | | | | | |
| Toru | Slow | Soil-borne | 1 | 4 | Tomato | 60 | 4 |
| Bam | seedling | disease | | | | | Labor/mu/year |
| | emergence | | | | | | |
| | and low | | | | | | |
| | germinat [symbol] | | | | | | |

[symbol]

indicates data missing or illegible when filed

Because the rootstock variety of eggplant is infertile and can not be propagated by self-crossing, it must be propagated rapidly through tissue culture. Therefore, after obtaining the regenerated buds, the rootstock population can be rapidly propagated through multiple clusters of buds and tissue culture.

Specifically, the embodiments of the present disclosure also provide a method for rapid propagation of the tree eggplant having the characteristics of fast growth, strong growth, good resistance, perennial and pest-resistant, comprising the following steps:

subculturing and propagating the regenerated shoots obtained from the hybridization; cutting and transferring the stem segments of the multi-cluster shoots of sterile seedlings to the multiplication and propagation medium (MS+2 mg/l ZR); expanding the cut ends of the stem segments into green dense tissue, and the buds grow out in the later stage, and further grow into plants; adding the MS medium with agar 7 g, sucrose 25 g, pH is 5.8 to 6.0, culture temperature 25±1° C., light intensity 25 μmol·m⁻²·s⁻¹ for 12 hours/day, and obtaining the regenerated sterile shoots after 10 days of culture.

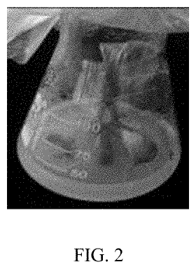

Cutting and transferring the regenerated sterile buds to a sealed tissue culture flask containing sterile bud rooting medium (½ MS+IBA 0.4 mg/l); FIG. 2 is a photograph of a tree eggplant obtained by rapid propagation of stem segments provided in an embodiment of the present disclosure, which is placed under natural scattered light, gradually opening the cap after 6 to 7 days, and refining the seedling for 3 days; and cleaning the culture medium on the root system with clean water, and then planting the seedling into the growth substrate mixed with the autoclaved organic fertilizer and perlite in a ratio of 1:1; pouring water, covering the film to keep moisture, putting the planted seedlings under the scattered light, paying attention to spraying the appropriate amount of water every day, gradually removing the film, and after the growth is relatively stable, gradually placing it under the strong light to grow.

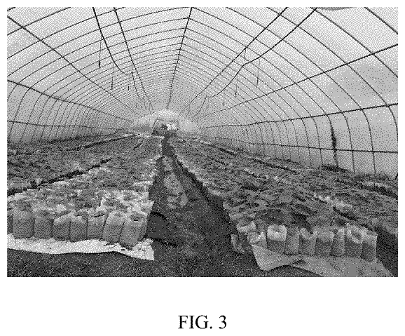

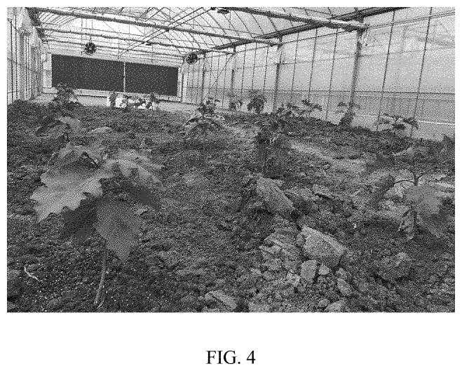

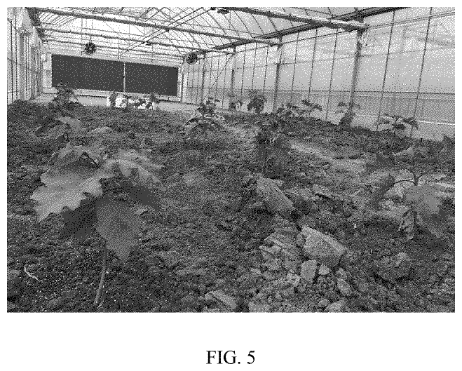

FIG. 3 and FIG. 4 are photographs of the propagation field of the seedlings obtained by rapid propagation provided in the embodiment of the present disclosure and the field photographs of the seedlings in the greenhouse; FIG. 5 is a photograph of the robust tree body obtained by rapid propagation provided in the embodiment of the present disclosure after pruning.

The obtained tree eggplant with fast growth, strong growth and good resistance can be used as rootstocks for grafting solanaceous plants, and can also be used as landscape trees for green planting in the tropical areas.

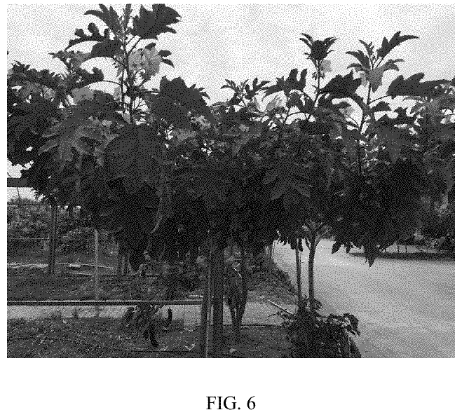

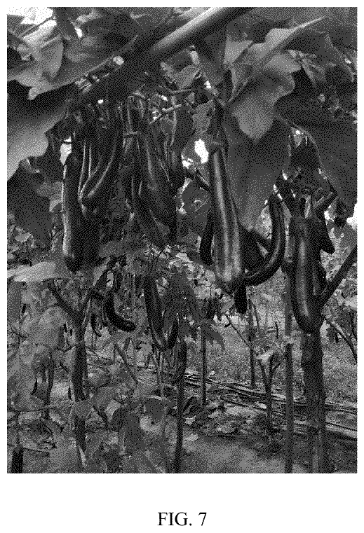

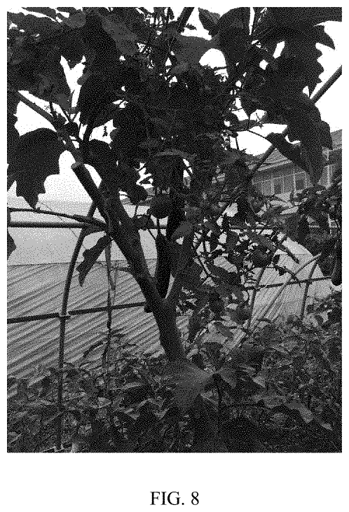

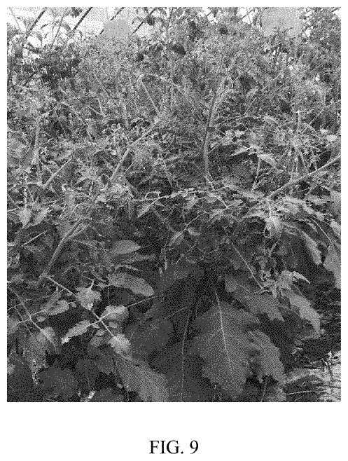

FIG. 6 is a field photograph of the tree eggplant provided in the embodiment of the present disclosure being planted in the green belt as a landscape tree species, and FIG. 7, FIG. 8 and FIG. 9 are field photographs of the tree eggplant provided in the embodiment of the present disclosure being used as a stock for high-position grafting of eggplant, being used as a stock for grafting eggplant and tomato at the same time, and being used as a stock for grafting tomato at the full flowering stage.

Finally, the method of the disclosure is only a preferred embodiment and is not intended to limit the scope of protection of the disclosure. Any modification, equivalent replacement, improvement, etc. made within the spirit and principles of the disclosure shall be covered by the protection of the disclosure.

What is claimed is:

1. A cultivation method of a tree eggplant, wherein:
taking wild a Solanum wrightii as a female parent, taking wild a Solanum torvum as a male parent, after hybrid pollination, and in turn after embryo rescue and seedling transplantation, obtaining the tree eggplant.

2. The cultivation method of the tree eggplant according to claim 1, wherein:
the hybrid pollination comprises: making emasculation and then bagging of a flower of the Solanum wrightii female parent 1 to 2 days before opening, selecting pollen of a male flower of the Solanum torvum male parent from 9:00 to 12:00 a.m. at a full opening stage of the male flower of the Solanum torvum male plant.

3. The cultivation method of the tree eggplant according to claim 1, wherein:
the embryo rescue comprises: taking an immature hybrid young fruit 20 to 25 days after the hybrid pollination, sterilizing with NaClO, cutting and isolating to obtain young embryo, and transferring to an induced proliferation medium for culture, then transferring induced embryoids to a regeneration medium to obtain regenerated buds, and finally transferring the obtained regenerated buds to a rooting medium to obtain rooting;
and/or, the seedling transplanting comprises washing a culture medium adhered to a test-tube seedling obtained by culturing and rooting with water, transplanting to seedling-refining substrate, periodically watering nutrient solution, covering with film and moisturizing, maintaining light, and performing the seedling transplanting after progressive conjunctiva.

4. The cultivation method of the tree eggplant according to claim 3, wherein:
the induced proliferation medium is MS+2 mg/l ZR, and a subculture temperature and a time are 22 to 28° C. and 30 to 40 days, respectively;
and/or, the regeneration medium is MS+2 mg/l 6-BA+0.5 mg/l IAA;
and/or, the rooting medium is MS+0.4 mg/l IBA for 20 to 36 days;
and/or, the seedling-refining substrate is a mixture of coconut bran, vermiculite and perlite in a volume ratio of (0.8-1.2):(0.8-1.2):(0.8-1.2).

5. The cultivation method of the tree eggplant according to claim 1, wherein: the tree eggplant with fast growth, strong growth and good resistance is obtained by the cultivation method of the tree eggplant.

6. A rapid propagation method of the tree eggplant according to claim 5, wherein:
comprising following steps:
S1: cutting stem segments with buds of aseptic seedlings of the tree eggplant, inoculating to a multiplication and propagation medium to induce proliferation, and obtaining multiplication and propagation buds;
S2: inoculating the multiplication and propagation buds into a tissue culture medium, then under conditions of 23 to 28° C., 22 to 27 μmol·m⁻²·s⁻¹ and 10 to 13.5 hours of sunlight, obtaining regenerated sterile buds after culturing for 10 to 14 days; and
S3: cutting the regenerated sterile buds and transferring to a sealed tissue culture flask containing a sterile bud rooting medium, placing under natural scattering light, then gradually opening to refine seedlings, and finally transferring to growth substrate for natural growth.

7. The rapid propagation method of the tree eggplant according to claim 6, wherein:
in the S1, the multiplication and propagation medium is MS+2 mg/l ZR, and a length of a shoot segment with the buds is 0.4 to 0.6 cm;
and/or, in the S2, the tissue culture medium is MS+7 g agar+25 g sucrose, pH is 5.8 to 6.0.

8. The rapid propagation method of the tree eggplant according to claim 7, wherein:
in the S3, the sterile bud rooting medium is ½ MS+IBA 0.2 mg/l, a seedling opening time is at least 3 days, and the growth substrate is a mixture of the perlite with a volume ratio of 1:1 and sterilized and decomposed organic fertilizer.

9. An application of the tree eggplant according to claim 5.

10. The application of the tree eggplant according to claim 9, wherein:
the application of the tree eggplant comprises being used as rootstocks for grafting solanaceous plant and perennial cultivation and as landscape trees for perennial growth in afforestation of tropical areas.
